(19) United States
(12) Patent Application Publication
(10) Pub. No.: US 2011/0178586 A1
(43) Pub. Date: Jul. 21, 2011

(54) CRANIAL ELECTROSTIMULATION ELECTRODE UNIT

(75) Inventor: Robert Russell Grey, Western Cape Province (ZA)

(73) Assignee: To Be First AG, Zug (CH)

(21) Appl. No.: 13/119,168

(22) PCT Filed: Sep. 17, 2009
(86) PCT No.: PCT/IB2009/006864

§ 371 Date: Mar. 15, 2011

(30) Foreign Application Priority Data
Sep. 18, 2008 (GB) 0817091.2

(51) Int. Cl. A61N 1/05

(52) U.S. Cl. 607/136

(57) ABSTRACT

An electrode unit is provided for use in cranial electrostimulation in which an electric current transferring electrode operatively transfers electrical energy to the region of an ear of a patient. The electrode unit has an electrode carried on the outer periphery of an audio earphone of the type suitable for engagement in the entrance to, or within, the auditory canal of an ear of a patient. The electrode has an electrical contact area for operatively transferring electrical energy between the electrode and the skin of a patient fitted with the electrode unit. The electrode may have a contact surface layer exposed for direct contact with the skin of a patient, in use, or it may have an electrically conductive cover for the electrode itself. Typically, the electrode unit forms one of a pair thereof in which instance the audio earphones are a pair of stereo earphones of substantially conventional type.

### FIELD OF THE INVENTION

This invention relates to an electrode unit for use in carrying out cranial electrostimulation especially, but not necessarily exclusively, electrostimulation in the form of what is known as cranial electro-biologic stimulation that is typically applied between electrodes clipped to the earlobes of a patient receiving treatment. Such a procedure is often referred to as electrotherapy in many of its applications.

It is to be understood that the term “patient” as used in this specification is intended to be interpreted broadly as being any person being subjected to electrostimulation irrespective of whether the underlying reason for applying such electrostimulation has curative intentions or not. In this regard it is noted that electrostimulation is often used with the intention of simply improving a patient's quality of life such as by improving the patient's mood, emotions, attitude or cognitive capabilities.

### BACKGROUND TO THE INVENTION

Cranial electro-biologic stimulation (herein referred to as CES) applies gentle micro-current pulses to the brain (in the hypothalamic area) using a pair of ear-clip electrodes that attach to the earlobes. It is widely accepted that CES stimulates the brain to manufacture neurotransmitters, like endorphins, which improve moods, emotions and cognitive capabilities. Cranial electro-stimulation has also been proposed for treatment following a stroke, brain trauma, certain heart disorders, high blood pressure, jet leg, motion sickness and dementia.

The signals apparently normalise the electrical output of the brain. CES has thus been used/tested to treat insomnia, substance dependence, depression and anxiety. It has been noted in at least some instances that CES has equal or greater efficacy for the treatment of depression when compared to antidepressant medications, with fewer side effects.

The mechanism by which CES produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

Treatments that have typically been used range from 10 to 30 minutes in duration although they may extend up to 1½ hours depending on the electrical current configuration. Typically the currents employed would be applied in pulse form with a pulse width in the range of from about 1 to about 500 milliseconds (ms) at a frequency of from about 0.1 Hertz (Hz) to about 500 Hz with the current being typically less than 1 milliampere (mA) and more typically about 300 to 700 microamperes (μA), although currents of up to about 2.5 milliampere (mA) have also been used.

The beneficial effects of music on the human body have long been appreciated and employed in the treatment, in particular, of psychological disturbances.

### OBJECT OF THE INVENTION

It is an object of this invention to provide an electrode unit for use in cranial electrostimulation that exhibits advantages over prior art ear lobe clips.

### SUMMARY OF THE INVENTION

In accordance with this invention there is provided an electrode unit for use in cranial electrostimulation, the electrode unit comprising an electric current transferring electrode for operatively transferring electrical energy to the region of an ear of a patient, the electrode unit being characterised in that the electrode is carried on the outer periphery of an audio earphone of the type suitable for engagement in the entrance to, or within, the auditory canal of an ear of a patient and wherein the electrode has an electrical contact for operatively transferring electrical energy between the electrode and the skin of a patient fitted with the electrode unit.

Further features of the invention provide for the electrical contact is a contact surface layer exposed for direct contact with the skin of a patient, in use, or alternatively, for the electrical contact to assume the form of an electrically conductive cover for the electrode itself; for the electrode to be carried on an electrically insulating surround to the audio earphone; for the electrode to be in the form of a ring surrounding the electrically insulating surround; and for the electrode unit to be arranged in pairs thereof in which instance the audio earphones may be a pair of stereo earphones of substantially conventional type.

It will be appreciated that the electrode units provided by the present invention can be used in carrying out any form of electrostimulation although it is generally envisaged that they will be most suitable for application in carrying out CES. Still more particularly, and whilst not limiting the invention in any way, it is envisaged that the electrode units of the invention will, inter alia, be particularly useful in the implementation of our co-pending patent application filed contemporaneously herewith under the title “CRANIAL ELECTROSTIMULATION METHOD AND EQUIPMENT” and claiming priority in terms if the International Convention of Paris from British Patent Application No 0817089.6. In that instance the electrostimulation pulses are generated at a frequency that is varied according to the rhythm, beat or tempo of music that is audible to the patient by way of the earphones.

In order that the above and other features of the invention may be more fully understood, different embodiments thereof will be described below with reference to the accompanying drawings.

### BRIEF DESCRIPTION OF THE DRAWINGS

In the drawings:—

FIG. 1 is a block diagram of one method and equipment for applying CES pulses by way of electrode units according to the invention;

FIG. 2 is a perspective illustration of a portable version of equipment operating on the basis of the block diagram of FIG. 1;

FIG. 3 is a perspective illustration of one embodiment of earphone according to the invention and that is suitable for use with the equipment illustrated in FIG. 2;

FIG. 4 is an exploded illustration thereof;

FIG. 5 illustrates in FIG. 5a an earphone about to be installed in an ear; in FIG. 5b the earphone installed; and in FIG. 5c the area of contact of the electrode with the ear; and,

FIG. 6 illustrates an alternative form of electrode unit according to the invention.

### DETAILED DESCRIPTION WITH REFERENCE TO THE DRAWINGS

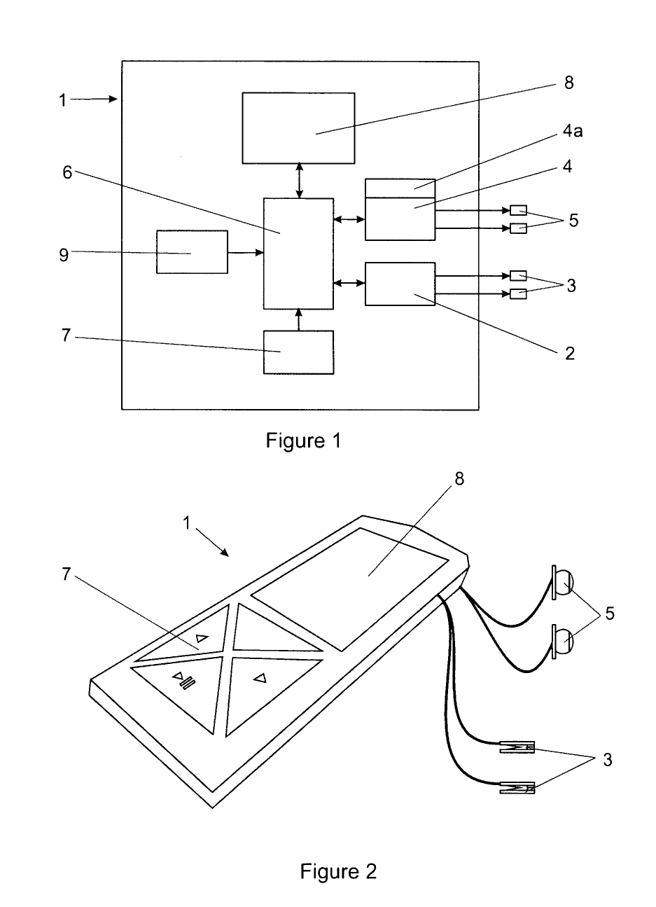

In the equipment illustrated in FIGS. 1 and 2, a portable battery powered unit (1) is employed to generate CES pulses to carry out CES therapy in a manner as set out in our co-pending patent application indicated above. The equipment may take the shape and configuration of a pocket sized therapy unit as illustrated in FIG. 2 or it may be a multi component arrangement such as for use in a treatment centre or studio.

The therapy unit is configured to carry out CES on a patient by the application of appropriate electrical pulses to the ears simultaneously with the playing of music that is audible to the patient. The equipment thus comprises a cranial electrostimulation pulse generator (2) and associated electrodes (3) for applying pulses generated by the pulse generator to the ears of a patient, and a sound signal generator (4) and a pair of earphones (5) for converting output from the sound signal generator into audible sound. The sound signal generator may have associated with it a data base containing a selection of different musical numbers that can be selected by a user or by selecting a predetermined treatment programme or session. The data base is indicated by numeral (4a) in FIG. 1.

The outputs from the electrostimulation pulse generator and sound signal generator are controlled by a controller in the form of a microprocessor (6) that may be user operated by a user button interface (7). The unit has a display screen (8) for assisting in programme selection in very much a conventional manner. Obviously the display screen could be replaced by any other suitable indicator means such as an array of light emitting diodes or the like. A battery charger circuit (9) is also provided.

The above arrangement is such that the electrostimulation pulse generator produces electrical pulses having a frequency and pulse width selected to provide a required microcurrent across the head, in use, such microcurrent typically being of the order of 500 to 700 μA and, in any event, less than 1 mA. The frequency could range from 0.5 Hz to about 3 Hz and the pulse width could range from about 125 ms at a frequency of 0.5 Hz to about 75 ms at a frequency of 3 Hz. The microcurrent across the head is, in any event, generally maintained at a substantially constant average current The pulses (or possibly sequences of pulses in the event that predetermined sequences of optionally different pulses are employed) are, in this instance, generated at a frequency that is dependant on the tempo of music that is being played at any one time and that is audible to the patient by way of the earphones. Of course, in order to achieve a substantially constant average current, the pulse width of the pulses will also vary with variation in frequency. The microprocessor monitors the tempo of the music and controls the generation of the electrical pulses on the basis thereof.

Whilst not in any way being limiting to the scope of the invention it is proposed that the following could operate effectively:—

At an audio tempo of 30 beats per minute a frequency of 0.5 Hz could be employed with a pulse width of 125 ms;
at an audio tempo of 60 beats per minute a frequency of 1.0 Hz could be employed with a pulse width of 115 ms;
at an audio tempo of 90 beats per minute a frequency of 1.5 Hz could be employed with a pulse width of 105 ms;
at an audio tempo of 120 beats per minute a frequency of 2.0 Hz could be employed with a pulse width of 95 ms;
at an audio tempo of 150 beats per minute a frequency of 2.5 Hz could be employed with a pulse width of 85 ms; and,
at an audio tempo of 180 beats per minute a frequency of 3.0 Hz could be employed with a pulse width of 75 ms.

The microprocessor is also programmed to provide treatment programmes or sessions of a predetermined duration, as is well known in the art, and the duration would generally be somewhere between 10 minutes and 30 minutes but may extend up to an hour or even an hour and a half. Information as to any particular treatment session to be chosen, musical numbers and the like can be displayed on the screen (8) provided on the therapy unit.

Reverting now to the nature of the earphones that are provided by this invention, the electrodes and earphones are combined into combination electrode units that fit into the entrance to the auditory canal of the ear.

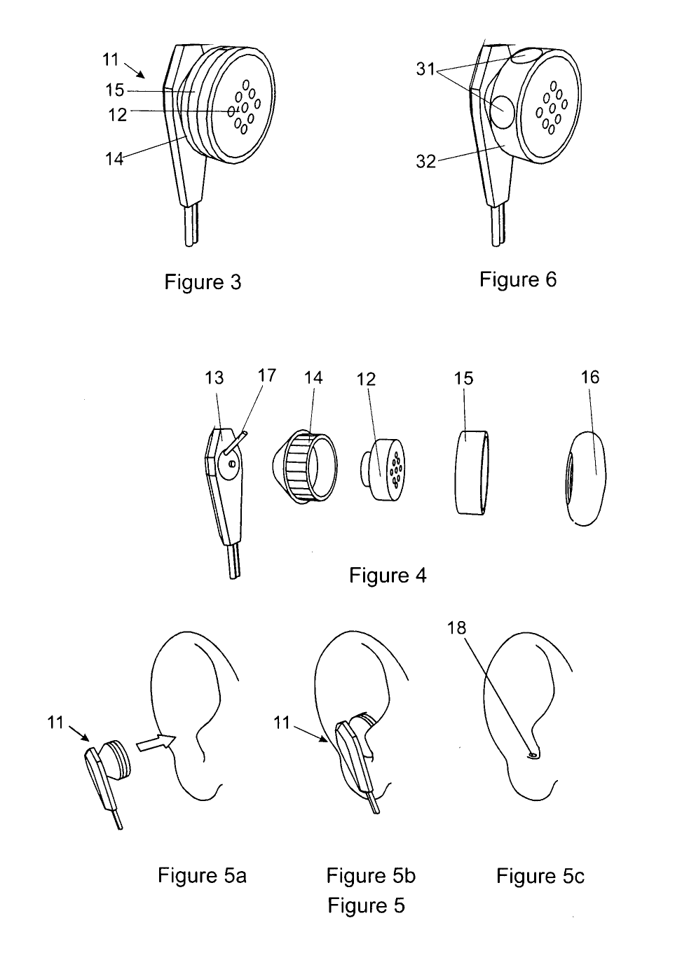

As illustrated in FIGS. 3 to 5 of the accompanying drawings, each earphone unit (11) may comprise a generally standard audio earphone (12) mounted on a base (13) and surrounded by an insulating ring (14). A ring-shaped electrode (15) surrounds the insulating ring and is covered by an electrically conductive cover in the form of an electrically conductive rubber sheath (16). The electrode is, of course, electrically connected to the electrostimulation pulse generator (2) by way of a suitable conductor (17) so that a current can be generated between the electrodes on two earphones contacting the two ears of a patient, in use.

As shown in FIG. 5, the earphone is adapted to be received in the entrance to the auditory canal so that the ring makes connection with the skin by way of the conductive rubber sheath, for example in the region indicated by numeral (18) in FIG. 5c. Of course, the earphone could be configured to be received within the auditory canal of the ear in the manner of what are often referred to canal phones or earbuds, as will be quite apparent to those skilled in the art.

FIG. 6 illustrates an alternative electrode unit in which the electrode (31) itself is in the form of a single “spot” electrode on a cylindrical insulating surface (32) of the type of earphone adapted to be received in the entrance to the auditory canal.

Of course the electrode unit provided by this invention can be used in association with any other type of CES treatment equipment and is in no way limited to use in association with that described above.

The use of a single electrode unit for both the application of electrical pulses to the ears of a patient and the simultaneous provision of music for a patient, whether or not the music is related in any way to the pulses, is extremely convenient and cost-effective. Such an electrode unit very much simplifies the conduct of electrostimulation accompanied by music for the patient to enjoy whilst undergoing the therapy and also simplifies setting up the equipment on a patient.

It will be understood that numerous variations may be made to the embodiments of the invention described above without departing from the scope hereof.

1. An electrode unit for use in cranial electrostimulation, the electrode unit comprising an electric current transferring electrode for operatively transferring electrical energy to the region of an ear of a patient, the electrode unit being characterised in that the electrode is carried on the outer periphery of an audio earphone of the type suitable for engagement in the entrance to, or within, the auditory canal of an ear of a patient and wherein the electrode has an electrical contact area for operatively transferring electrical energy between the electrode and the skin of a patient fitted with the electrode unit.

2. An electrode unit as claimed in claim 1 in which the electrical contact is a contact surface layer exposed for direct contact with the skin of a patient, in use.

3. An electrode unit as claimed in claim 1 in which the electrical contact is an electrically conductive cover for the electrode itself.

4. An electrode unit as claimed in claim 1 in which the electrode is carried on an electrically insulating surround to the audio earphone.

5. An electrode unit as claimed in claim 4 in which the electrode is in the form of a ring surrounding the electrically insulating surround.

6. An electrode unit as claimed in claim 1 in which the electrode unit forms one of a pair thereof in which instance the audio earphones are a pair of stereo earphones of substantially conventional type.
